(19) United States
(12) Patent Application Publication
(10) Pub. No.: US 2009/0318797 A1
(43) Pub. Date: Dec. 24, 2009

(54) SYSTEM AND METHOD FOR DEFLECTING ENDOSCOPIC TOOLS

(75) Inventor: Ron Hadani, Cresskill, NJ (US)

Correspondence Address: MARTIN D. MOYNIHAN d/b/a PRTSI, INC., P.O. BOX 16446, ARLINGTON, VA, 22215, US

(73) Assignee: Vision-Sciences Inc., Orangeburg, NY (US)

(21) Appl. No.: 12/486,795

(22) Filed: Jun. 18, 2009

Related U.S. Application Data
(60) Provisional application No. 61/129,344, filed on Jun. 19, 2008.

(51) Int. Cl. A61B 1/018; A61B 5/05; G06K 9/00; G08B 7/00

(52) U.S. Cl. 600/424; 600/106; 382/103; 340/691.5

(57) ABSTRACT

An endoscope sheath, comprising:
an endoscope housing, configured for housing at least a distal end of an insertion tube of an endoscope and allowing the traversing of an endoscopic tool therethrough; and
a tool deflector configured for deflecting said endoscopic tool by changing a direction of movement of said endoscopic tool in relation to a direction of movement of said distal end of said insertion tube.

### RELATED APPLICATION

This application claims the benefit under 35 USC 119(e) of priority of U.S. Provisional Patent Application No. 61/129,344, filed on Jun. 19, 2008, the contents of which are incorporated by reference as if fully set forth herein.

### FIELD AND BACKGROUND OF THE INVENTION

The present invention, in some embodiments thereof, relates to medical devices and, more particularly, but not exclusively, to catheters, endoscopes, endoscopic tools, and minimally invasive probes. Some embodiments of the present invention relate to deflecting an endoscopic tool associated with an endoscope. Some embodiments of the present invention relate to the tracking of endoscopic tools within a body lumen.

Endoscopy is a minimally invasive diagnostic medical procedure that is used to assess the interior surfaces of an organ by inserting an insertion tube of an endoscope into the body. A typical endoscope includes a rigid or flexible insertion tube and an endoscope control unit, such as a handle, for allowing a user to hold and/or control the insertion tube, to manipulate the insertion tube in the body, and to control video functions such as image capture and image freeze frame. The insertion tube is usually designed to provide an image of the body lumen for visual inspection and photography, which may be performed by a variety of image capturing devices. A frequently-used image capturing device is the ultrasound imager. Endoscopy is also a vehicle for minimally invasive surgery.

Minimally invasive surgical procedures avoid open invasive surgery in favor of closed or local surgery with fewer traumas. These procedures involve remote-control manipulation of endoscopic tools with observation of the surgical field through an endoscope or similar device, and are carried out through the skin or through a body cavity or anatomical opening. Endoscopic tools are elements configured for treating and/or probing targets in body lumens. There exist many kinds of endoscopic tools, each endoscopic tool having a specific function or a limited number of functions. Common examples of endoscopic tools are needles, used for injecting substances into target tissues or obtaining a tissue sample, biopsy forceps, used to remove one or more tissue samples for analysis, and endoscopic graspers, configured for grasping slippery tissue or foreign bodies. Endoscopic tools are also referred to as “tools”, “medical tools” or “endoscope tools” in the art.

Endoscopic tools used in minimally invasive surgery reach a target area within a body cavity or lumen and perform some function to it. Endoscopic tools are usually associated with endoscopes, are inserted into working channels within the insertion tube portions of the endoscope, and move either within the working channels or with the insertion tubes as they move inside the body lumen. Some endoscopic tools only move with the insertion tube they are associated with. Other endoscopic tools are equipped with elements that allow them some limited movement, independent of the insertion tube. These endoscopic tools can be controlled and moved with a certain degree of freedom, and are not completely dependent on the movement of the insertion tube.

Elements that allow the movement of endoscopic tools by changing the orientation of the tool with respect to the orientation of the insertion tube are herein called tool deflectors. In the art, tool deflectors are also referred to as “deflectors”. Two kinds of tool deflectors are commonly used: cam type deflectors, and electromechanical deflectors.

A cam type deflector is an element placed near the distal end of the insertion tube of an endoscope, and is in direct physical contact with the endoscopic tool. When the endoscopic tool is slightly pushed in or pulled out of the body lumen, the distal end of the tool slips against the cam type deflector, and the orientation of the tool is changed. Cam type deflectors may be fixed or movable.

An electromechanical deflector is a moving element in contact with the distal end of the tool. The electromechanical deflector is controlled by electrical signals and may be moved along many axes, in order to change the orientation of the tool. An electromechanical deflector available in the market is the Olympus TJF type 160VF.

### SUMMARY OF THE INVENTION

The present invention, in some embodiments thereof, relates to medical devices and, more particularly, but not exclusively, to catheters, endoscopes, endoscopic tools, and minimally invasive probes. Some embodiments of the present invention relate to deflecting an endoscopic tool associated with an endoscope. Some embodiments of the present invention relate to the tracking of endoscopic tools within a body lumen.

According to an aspect of some embodiments of the present invention, there is provided an endoscope sheath, including an endoscope housing, configured for housing at least a distal end of an insertion tube of an endoscope and allowing the traversing of an endoscopic tool therethrough, and a tool deflector configured for deflecting the endoscopic tool by changing a direction of movement of the endoscopic tool in relation to a direction of movement of the distal end of the insertion tube.

According to some embodiments of the invention, the insertion tube belongs to a bronchoscope.

According to some embodiments of the invention, the endoscope housing is configured for housing the whole endoscope. According to some embodiments of the invention, the endoscope sheath is configured for being used only once.

According to some embodiments of the invention, the endoscopic tool traverses the endoscope housing through at least one opening located on a surface of the endoscope housing.

According to some embodiments of the invention, the opening is located at a distal extremity of a sheath channel, the sheath channel being configured for housing at least a portion of the endoscopic tool.

According to some embodiments of the invention, the tool deflector deflects the endoscopic tool in a plurality of directions. According to some embodiments of the invention, the tool deflector is located on a surface of the endoscope housing, between a point at which the endoscopic tool traverses the endoscope housing and a distal extremity of the endoscope housing.

According to some embodiments of the invention, the tool deflector is located on one of an inner surface of the endoscope housing, and an outer surface of the endoscope housing.

According to some embodiments of the invention, the sheath channel is an integral part of the endoscope housing, and protrudes from the endoscope housing. According to some embodiments of the invention, the sheath channel is detachable from the endoscope housing.

According to some embodiments of the invention, the tool deflector is configured to change the orientation of the endoscopic tool by moving a distal end of the sheath channel.

According to some embodiments of the invention, the tool deflector is inflatable and deflects the distal end of the endoscopic tool by expanding and contracting.

According to some embodiments of the invention, the endoscope sheath further includes a conduit for moving at least one fluid into and out of the tool deflector.

According to some embodiments of the invention, a fluid within the tool deflector is heated to expand, and cooled to contract, thereby expanding and contracting the tool deflector or a portion of the tool deflector.

According to some embodiments of the invention, the point at which the sheath housing is traversed by the endoscopic tool is positioned such that at least one tip of the endoscopic tool is within a field of view of an imaging sensor attached to a distal end of the insertion tube.

According to some embodiments of the invention, the imaging sensor is selected from a group of an ultrasound imaging sensor, a charged couple device (CCD) sensor, and a complementary metal oxide semiconductor (CMOS) sensor, a magnetic endoscopic imager (MEI) sensor, and a fluorescent imager.

According to an aspect of some embodiments of the present invention, there is provided an endoscope, including an endoscopic channel, traversing an insertion tube of the endoscope, and configured for housing at least one endoscopic tool, and an inflatable tool deflector located at a distal end of the endoscopic channel, for gradually deflecting a distal end of the endoscopic tool in relation to a distal end of the insertion tube, wherein the inflatable tool deflector is in contact with the endoscopic tool, and a change in at least one property of the inflatable tool deflector causes a deflection of the distal end of the endoscopic tool, with respect to the distal end of the insertion tube, by changing a direction of movement of the distal end of the endoscopic tool in relation to a direction of movement of the distal end of the insertion tube.

According to some embodiments of the invention, the above endoscope is a bronchoscope configured for being inserted into the airways of a patient.

According to some embodiments of the invention, the property of the inflatable tool deflector is one of volume and shape.

According to some embodiments of the invention, the endoscope further includes at least one imaging sensor, for providing an image of at least a body lumen within which the insertion tube is inserted.

According to some embodiments of the invention, the imaging sensor is selected from a group of an ultrasound imaging sensor, a charged couple device (CCD) sensor, and a complementary metal oxide semiconductor (CMOS) sensor, a magnetic endoscopic imager (MEI) sensor, and a fluorescent imaging sensor.

According to some embodiments of the invention, a distal tip of the endoscopic tool housed in the endoscopic channel is within a field of view of the imaging sensor, so that an image of the distal tip is provided by the imaging sensor.

According to some embodiments of the invention, the endoscope is configured to be connected to an image processor, which determines the orientation of the endoscopic tool, through an analysis of the image of the tip.

According to an aspect of some embodiments of the present invention, there is provided a system for generating a trajectory track of an endoscopic tool associated with an endoscope within a body lumen, including at least one imaging sensor attached to a distal end of an insertion tube of the endoscope, for generating an image of the body lumen, a processing unit, for calculating the trajectory of the endoscopic tool based on an orientation of the endoscopic tool, and a screen, for displaying the trajectory track on the image.

According to some embodiments of the invention, the image of the body lumen further includes a tip on a distal side of the endoscopic tool.

According to some embodiments of the invention, the above system further includes an image processing unit, for calculating the orientation of the endoscopic tool, based on the image from the imaging sensor.

According to some embodiments of the invention, the imaging sensor is selected from a group of an ultrasound imaging sensor, a charged couple device (CCD) sensor, and a complementary metal oxide semiconductor (CMOS) sensor, a magnetic endoscopic imager (MEI) sensor, and a fluorescent imaging sensor.

According to some embodiments of the invention, the above system further includes a tool deflector, for changing the orientation of the endoscopic tool, relative to the endoscope, and a measuring unit, for measuring at least one property of the tool deflector, combined so that the processing unit receives the measured property from the measuring unit, and calculates the orientation of the endoscopic tool, according to the property.

According to some embodiments of the invention, the tool deflector includes at least one inflatable unit, and the measuring unit measures one or more of a flow of fluid into and out of the inflatable unit of the tool deflector, a pressure of the fluid, and a volume of the fluid within the tool deflector.

According to some embodiments of the invention, the one imaging sensor generates the image that further includes the target. According to some embodiments of the invention, the above system is configured for guiding an endoscopic tool associated to an endoscope toward a target within a body lumen, and further includes including a tool deflector, for changing the orientation of the endoscopic tool toward the target.

According to an aspect of some embodiments of the present invention, there is provided a method for changing an orientation of an endoscopic tool associated with the endoscope, relative to a distal end of an insertion tube of the endoscope, including inserting the insertion tube into a body lumen, and changing the orientation of the endoscopic tool by changing at least one property of an inflatable tool deflector in contact with the endoscopic tool.

According to some embodiments of the invention, the above property is one of volume and shape.

According to some embodiments of the invention, the above method further includes providing an image of a target within the body lumen, and calculating the orientation of the endoscopic tool.

According to some embodiments of the invention, the calculating is performed through at least one of an analysis of an image of the endoscopic tool, by an image processor, and a measurement of at least one property of at least one fluid directed to the inflatable tool deflector, and a conversion of the property into the orientation, according to calibration data.

According to some embodiments of the invention, the above method further includes calculating an estimated trajectory of the endoscopic tool, based on the orientation, and superimposing a graphical trajectory track of the trajectory on the image.

According to some embodiments of the invention, the above method further includes changing the orientation of the endoscopic tool so that the graphical trajectory track crosses the image of the target, advancing the insertion tube and/or the endoscopic tool farther into the body lumen, observing whether the graphical trajectory track crosses the image of the target, and changing the orientation of the endoscopic tool, if needed.

According to some embodiments of the invention, the observing is performed by a processing unit, and further including one or more of assigning a color to the graphical trajectory track, according to the orientation of the graphical trajectory track relative to the image of the target, and emitting a sound, according to the orientation of the graphical trajectory track relative to the image of the target.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1A is a schematic drawing illustrating an endoscope sheath characterized by an opening and featuring an inflatable tool deflector, according to some embodiments of the invention;

FIG. 1B is a schematic drawing illustrating an endoscope sheath comprising a tool deflector, an endoscope housing, and a tool channel external to and independent of the endoscope housing, according to some embodiments of the invention;

FIG. 2 is a schematic drawing illustrating an endoscope sheath covering the whole endoscope, according to some embodiments of the invention;

FIG. 3 is a schematic drawing illustrating an endoscope sheath featuring a sheath channel for housing an endoscopic tool, according to some embodiments of the invention;

FIGS. 4a and 4b are schematic drawings illustrating an insertion tube of an endoscope, featuring a tool deflector located at a distal end of an endoscopic channel, according to some embodiments of the invention;

FIG. 5 is a schematic drawing illustrating a system for guiding an endoscopic tool associated with an endoscope toward a target within a body lumen, with the help of image processing, according to a preferred embodiment of the invention;

FIG. 6 is a schematic drawing illustrating a system for guiding an endoscopic tool associated with an endoscope toward a target within a body lumen, without the help of image processing, according to an alternative embodiment of the invention; and

FIG. 7 is a flowchart illustrating a method for guiding an endoscopic tool associated with an endoscope toward a target within a body lumen, according to some embodiments of the invention.

### DESCRIPTION OF SPECIFIC EMBODIMENTS OF THE INVENTION

The present invention, in some embodiments thereof, relates to medical devices and, more particularly, but not exclusively, to catheters, endoscopes, endoscopic tools, and minimally invasive probes. Some embodiments of the present invention relate to deflecting an endoscopic tool associated with an endoscope. Some embodiments of the present invention relate to the tracking of endoscopic tools within a body lumen.

Before defining any elements, units, apparatuses, and systems included in the present invention, a few important terms are to be defined. The term “distal”, when referred to an object, refers to a part of the object, which is farthest from a user and closest to a target within the body lumen. The term “proximal”, when referred to an object, conversely, refers to a part of the object, which is closest to a user and farthest from a target within the body lumen.

The term “end”, when referred to an object, refers to a part of the object which spans the last few centimeters before an extremity of the object. The term “tip”, when referred to an object, refers to a smaller part of the object which generally spans the last few millimeters before an extremity of the object.

An aspect of some embodiments of the present invention relates to a system for changing the orientation of an endoscopic tool, with respect to a direction of movement of a distal end of an insertion tube of an endoscope to which the endoscopic tool is associated. Embodiments of the invention include, but are not limited to, an endoscope sheath including a tool deflector, and an endoscope including a tool deflector.

An aspect of some embodiments of the present invention relates to an endoscope sheath. The endoscope sheath features a tool deflector configured for changing the orientation of an endoscopic tool in a body lumen. The endoscope sheath includes an endoscope housing, which is configured for housing at least an insertion tube of an endoscope and for being traversed by an endoscopic tool, for example through one or more openings on the endoscope sheath, and a tool deflector.

According to some embodiments of the present invention, the endoscope housing is configured for housing at least an insertion tube of a bronchoscope. Optionally the insertion tube is rigid, and configured, for example, for removing objects that have become obstructed in the airways of a patient. Optionally, the insertion tube is flexible, and configured, for example, for reaching remote areas within the airways and probing and/or treating the above areas.

The endoscope housing covers at least a distal end of the insertion tube inserted inside a body lumen or cavity, to promote sterility. This may allow the endoscope to be reused without performing cumbersome sterilization or high level disinfection procedures. Optionally, the endoscope housing is thin-walled, elongate, tubular, and made out of flexible material. Optionally, the endoscope housing covers the whole length of the insertion tube, from the point of insertion within the body lumen to the distal extremity of the insertion tube. Optionally, the endoscope housing covers the whole endoscope, including the handle.

Optionally, the endoscope sheath is configured for being used only once. Such a configuration enhances the sterility of endoscopy and minimally invasive surgery, as a new and sterile endoscope sheath is used for each procedure to cover at least part of the endoscope.

Optionally, the surface of the endoscope sheath is characterized by one or more openings configured for being traversed by one or more endoscopic tools. Optionally, the opening is located at a distal end of the endoscopic housing. Optionally, the opening is located at the distal extremity of a sheath channel, the sheath channel being an extension of the endoscope sheath, and being configured to cover part of the endoscopic tool. The distal tip of the endoscopic tool is not housed within the sheath channel, since the tip needs to come in direct contact with specific zones within the body lumen which are treated and/or probed by the endoscopic tool. Optionally, the sheath channel is distinct from the endoscope housing of the sheath. Optionally, the sheath channel is an integral part of the endoscope housing, protruding from the endoscope housing. Optionally, the sheath channel is a detachable element that may be fitted onto the endoscope housing, according to a user's need.

The tool deflector is used to change the orientation of the endoscopic tool. Optionally, the tool deflector is mounted at the distal end the endoscope housing. Optionally, the tool deflector is located on an outer surface of the endoscopic sheath. Optionally, the tool deflector is located on an inner surface of the endoscopic sheath. Optionally, the tool deflector changes the orientation of the tool through direct contact with the endoscopic tool. Optionally, according to a preferred embodiment of the invention, the tool deflector is placed between the endoscope sheath and the sheath channel, and changes the orientation of the endoscopic tool, by shifting the distal end of the sheath channel in which the endoscopic tool is housed. Placing the sheath channel between tool deflector and endoscopic tool decreases the chances of tool deflector damage that may be caused by the direct contact between sharp edges of the endoscopic tool and the tool deflector.

Optionally, according to a preferred embodiment of the invention, the tool deflector includes an inflatable unit, which expands when fluid, either liquid or gas, is directed into the inflatable unit. The expansion and contraction of the tool deflector's surface change the orientation of the endoscopic tool. Optionally, the tool deflector includes two or more inflatable units, each of which may expand or contract individually when fluid is directed into or out of the individual inflatable units. Optionally, the tool deflector is an electromechanical deflector, such as the Olympus TJF type 160VF model described above. Optionally, the tool deflector is a cam-type deflector, described above.

Some embodiments of the present invention relate to an endoscope which includes a tool deflector configured to change the orientation of an endoscopic tool in a body lumen. The endoscope includes an endoscopic channel and an inflatable tool deflector placed at the distal end of the endoscopic channel. Optionally, the endoscope is configured to hold one or more imaging sensors, for example, an ultrasound imaging sensor.

The endoscopic channel is an open lumen which traverses the insertion tube of the endoscope along the length of the insertion tube, from a proximal extremity thereof to a distal extremity thereof, and is configured to house an endoscopic tool. The tool deflector changes the orientation of the endoscopic tool. Optionally, the tool deflector is within the endoscopic channel, at the distal end of the endoscopic channel. Optionally, the tool deflector is right outside the distal extremity of the endoscopic channel. In either case, the tool deflector is in contact with the endoscopic tool. The tool deflector is made of an inflatable material, and expands when fluid (liquid or gas) is directed into the tool deflector. The expansion and contraction of the tool deflector change the orientation of the endoscopic tool. The tool deflector may comprise two independently inflatable chambers operable to deflect the endoscopic tool in two different directions.

Another aspect of some embodiments of the present invention relates to a system and a method for guiding an endoscopic tool associated with an endoscope toward a target within a body lumen. The system includes a tool deflector, one or more imaging sensors, and a processing unit.

An imaging sensor, for example an ultrasound imaging sensor, is attached to a distal end of the endoscope's insertion tube. The imaging sensor produces an image of the body lumen, or the tissue surrounding the body lumen, the image including the target that needs to be reached by the endoscopic tool. A processing unit calculates the trajectory of the endoscopic tool according to the orientation of the endoscopic tool. A screen displays the image of the lumen and a graphical trajectory track of the endoscopic tool. The tool deflector is configured to be controlled by a user, and allows the user to change the orientation of the endoscopic tool so that the graphical trajectory track displayed on the screen crosses the target. Once the trajectory track crosses the target, the user moves the insertion tube and/or the endoscopic tool farther inside the lumen, toward the target. Adjustments may be made in real time, if the endoscopic tool deviates from its desired trajectory.

According to a preferred embodiment of the invention, the image further includes a distal tip of the endoscopic tool, and the apparatus further includes an image processor, that calculates the orientation of the endoscopic tool, based on the image that contains the distal tip of the endoscopic tool.

According to an alternative embodiment of the invention, the above system uses a measuring unit for calculating the orientation of the medical tool. For example, the measuring unit measures one or more properties of the tool deflector. A calibration procedure is performed for determining the orientation of the endoscopic tool as a function of the above one or more properties of the tool deflector. The measuring unit sends the measured one or more properties to the processing unit, which calculates the orientation of the endoscopic tool, according to the above calibration.

Before explaining at least one embodiment of the invention in detail, it is to be understood that the invention is not necessarily limited in its application to the details of construction and the arrangement of the components and/or methods set forth in the following description and/or illustrated in the drawings. The invention is capable of other embodiments or of being practiced or carried out in various ways.

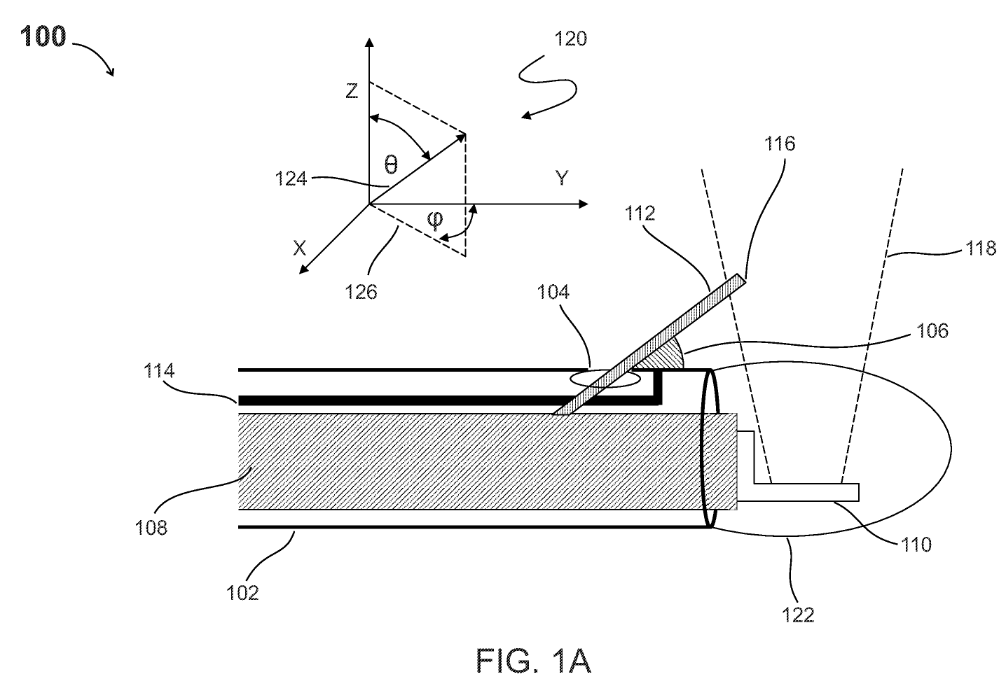

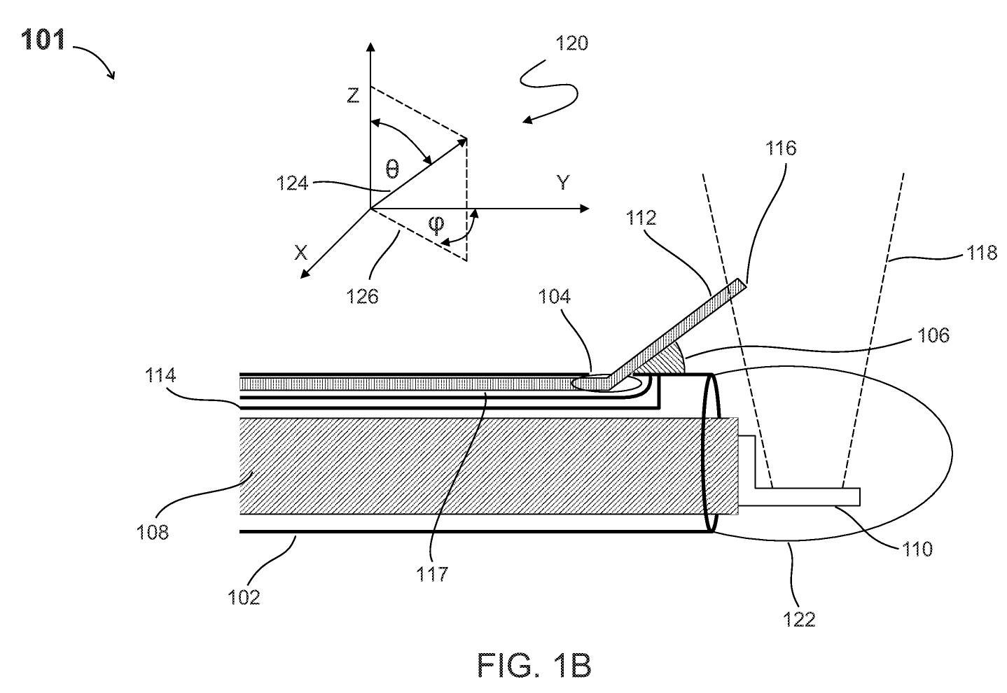

Referring now to the drawings, FIG. 1A is a schematic drawing illustrating an endoscope sheath featuring an inflatable tool deflector, according to a preferred embodiment of the invention.

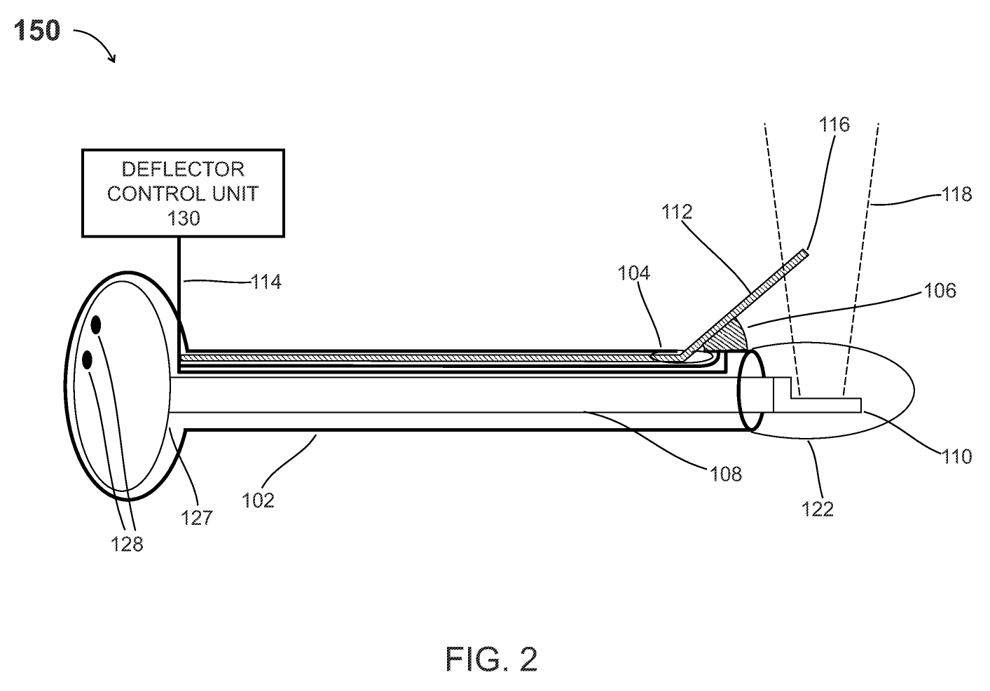

The endoscope sheath of apparatus 100 includes a tool deflector 106, and an endoscope housing 102. Endoscope housing 102 is configured to house at least a distal end of an insertion tube 108 belonging to an endoscope, and for being traversed by at least an endoscopic tool 112. Optionally, insertion tube 108 belongs to a bronchoscope. Optionally, endoscope housing 102 is configured for housing a length of insertion tube 108, starting from a point of insertion of insertion tube 108 within a body lumen to a distal extremity of insertion tube 108. Optionally, endoscope housing 102 is configured to house the whole endoscope, as shown in FIG. 2.

Optionally, endoscope housing 102 features at least one opening 104, configured for being traversed by endoscopic tool 112. Endoscope tool 112 may be a component of or attached to insertion tube 108, or may be an independent tool advanced through a working channel within insertion tube 108. Tool deflector 106 is configured for changing an orientation of endoscopic tool 112. Optionally, tool deflector 106 is located on an inner surface of endoscope housing 102. Optionally, tool deflector 106 is located on an outer surface of endoscope housing 102. Optionally, insertion tube 108 is configured to hold an imaging sensor 110 at a distal end of insertion tube 108. Optionally, insertion tube 108 is configured to hold more than one imaging sensor. Optionally, insertion tube 108 includes optical fibers, as commonly found in bronchoscopes, for conveying an image of the body lumen to a user. Optionally, insertion tube 108 holds a light source—for example a light emitting diode (LED)—in order to illuminate the body lumen and improve the quality of the image seen by the user.

Optionally, imaging sensor 110 is enclosed within a balloon 122, which is located at a distal extremity of endoscope housing 102. This enhances the isolation between the endoscope and the body lumen. Optionally, balloon 122 is an integral part of the endoscope sheath. Optionally, balloon 122 is detachable from the endoscope housing 102. Optionally, the interior of balloon 122 is filled with an ultrasonically transmissive medium, in order to create an ultrasonic circuit between imaging sensor 110 and a tissue to be scanned without the need to fill the whole body lumen with the transmissive medium. Optionally, balloon 122 is filled with the transmissive medium before insertion tube 108 is inserted in the body lumen. Optionally, the transmissive medium is directed to the interior of balloon 122, while insertion tube 108 is inside the body lumen. Optionally, the transmissive medium is one, more, or a combination of water, saline solution, and gel.

Endoscope housing 102 may be substantially rigid or substantially flexible. Endoscope housing 102 is optionally made out of polyurethane and/or PVC.

Imaging sensor 110 is configured to provide an image of the body lumen within which the endoscope is inserted. Optionally, imaging sensor 110 is an ultrasound imaging sensor. Optionally imaging sensor 110 is a charged couple device (CCD) sensor. Optionally, imager 110 is a complementary metal oxide semiconductor (CMOS) sensor. Optionally, imaging sensor 110 is a magnetic endoscopic imager (MEI) sensor, or a fluorescent imager sensor. It should be noted that insertion tube 108 may be configured to hold any kind of imaging sensor, or multitude of sensors, and that the choice of imaging sensor does not affect the functionality of the present invention.

Optionally, tool deflector 106 includes one or more inflatable units. Optionally, the inflatable unit or units are made of one or more thermoplastic elastomers, such as polyurethane. Optionally, the inflatable unit or units are made of one or more reinforced elastomers. As the inflatable units of tool deflector 106 expand or contract, and therefore change volume and/or shape, the orientation of the distal end of endoscopic tool 112 changes, by direct contact with endoscopic tool 112 with tool deflector 106. Optionally, inflatable unit or units of tool deflector 106 are inflated and deflated with a gas, such as air, carbon dioxide or nitrogen. Optionally, tool deflector 106 is inflated and deflated with a liquid, such as water or saline solution.

Optionally, the endoscope sheath of embodiment 100 further includes a conduit 114 attached to tool deflector 106 and used for moving fluid (liquid or gas) into and out of tool deflector 106. Optionally conduit 114 is configured to be connected to a deflector control unit, for controlling the inflation and deflation of tool deflector 106. Optionally conduit 114 is subdivided into a plurality of sub-conduits for independently controlling inflation and deflation of independent inflation chambers of deflector 106. Optionally, the deflector control unit comprises one or more piston-cylinder devices. Optionally, the piston or pistons are driven by a user through electronically controlled linear actuators. Optionally the piston-cylinder device or devices are syringes. Optionally, the syringe or syringes include a barrel characterized by screw threads, which allow a user to rotate the barrel in and out of the syringe's cylinder, with a high degree of precision. Optionally, conduit 114 is configured to be connected to a variable pressure regulator for controlling the inflation and deflation of tool deflector 106. For example, the variable pressure regulator may be of a common type where there is a spring loaded diaphragm, in which a pressure exerted by the spring on the diaphragm dictates a pressure of the fluid flowing through conduit 114. Variable pressure regulators of this type are manufactured, for example, by Watts Regulator Company, and Fairchild Industrial Products Company.

Optionally, sub-conduits of conduit 114 are each connected to a variable pressure regulator for controlling the inflation and deflation of individually inflatable units of tool deflector 106, providing for tool deflection in more than one plane.

Optionally, before the insertion of insertion tube 108 into the body lumen, tool deflector 106 is filled with a fluid. Within the body lumen, tool deflector 106 (or individually inflatable sub-units thereof) is expanded and contracted by expanding and compressing the fluid within tool deflector 106. For example, the fluid within an inflatable unit of tool deflector 106 may be expanded and compressed through a heating and cooling of the fluid. For example, a thermoelectric unit may be enclosed within a unit of tool deflector 106, and controlled to change the temperature of the fluid. Optionally, the thermoelectric unit includes one or more resistors, for heating the fluid. Optionally, the thermoelectric unit includes one or more Peltier thermoelectric devices, for heating and cooling the fluid.

Optionally, tool deflector 106 comprises one or more electromechanical deflectors, for example as described in the background section. Optionally, tool deflector 106 comprises one or more cam type deflectors, for example as described in the background section.

In apparatus 100, the endoscope sheath is configured so that a distal tip 116 of endoscopic tool 112 is within a field of view 118 of imaging sensor 100. Optionally, distal tip 116 of endoscopic tool 112 is not within field of view 118.

Tool deflector 106 gives endoscopic tool 112 a certain degree of freedom, and decreases the dependency of endoscopic tool 112 on the movement of insertion tube 108. For example, tool deflector 106 allows a change in the orientation of endoscopic tool 112, independent of the movement of insertion tube 108. Furthermore, if insertion tube 108 is placed close enough to a target to be treated and/or probed by endoscopic tool 112, tool deflector 106 may also allow endoscopic tool 112 to reach the target without any further movement by insertion tube 108.

Referring now to a coordinate system 120, Y is defined to be the direction of movement of a distal end of insertion tube 108, and X and Z are constructed to be normal to Y and to each other. Direction 124 is an orientation of endoscopic tool 112. The orientation of endoscopic tool 112 is defined by an angle θ formed between direction 124 and the Z axis, and an angle φ formed between a projection 126 of direction 124 upon the XY plane and the Y axis. Tool deflector 106 changes the direction of movement of endoscopic tool 112, by changing either angle θ (e.g. using a first inflatable unit, or a first electromechanical or cam deflector), angle φ (e.g. using a second inflatable unit, or electromechanical or cam deflector), or a both angles. Alternatively, direction 124 may be changed by rotating tube 108 within the body conduit.

The inclusion of tool deflector 106 in apparatus 100 allows the use of endoscopic tools that do not possess means for changing orientation. Such endoscopic tools are more common and less expensive than endoscopic tools with means for changing orientation.

FIG. 1B a schematic drawing illustrating an endoscope sheath 101 (also called “apparatus 101”) which comprises an endoscope housing 102 and a tool channel 117, according to an embodiment of the invention. Tool channel 117 functions as a “working channel”, a conduit through which a tool 112 may be advanced alongside endoscope 108. In some embodiments, tool channel 117 is external to and independent of housing 102, though optionally they are connected. Accordingly, in some embodiments tool 112, which comes in contact with body tissues, does not come in contact with endoscope insertion tube 108 inserted in housing 102. Aside from this difference, all sheath features and tool-directing features and other features described above with reference to apparatus 100 may also be available in sheath 101, and their descriptions will not be repeated here.

FIG. 2 is a schematic drawing illustrating an endoscope sheath with an endoscope housing 102 that covers the whole endoscope, according to some embodiments of the invention. In FIG. 2, Apparatus 150 includes the same elements as apparatus 100 of FIG. 1A or of sheath 101 of FIG. 1B. In FIG. 2, an endoscope is shown. The endoscope includes an insertion tube 108 and an endoscopic handle 127. Optionally, endoscopic handle 127 allows a user to control and/or to maneuver the operation of endoscopic tool 112, for example through a user interface (UI), as shown at 128. Optionally, endoscopic handle 127 also allows the user to control the operation of imaging sensor 110.

Optionally, conduit 114, as described above, is connected to deflector control unit 130. Optionally, deflector control unit 130 comprises one or more piston-cylinder devices, as described above.

In the embodiment illustrated by FIG. 2, the whole endoscope is covered by endoscope housing 102. Such an embodiment enhances the isolation of the endoscope from the surroundings, and therefore may allow the endoscope to be reused without performing cumbersome sterilization or high level disinfection procedures. Alternatively, housing 102 may cover only that portion of endoscope 108 which is introduced into a patient's body.

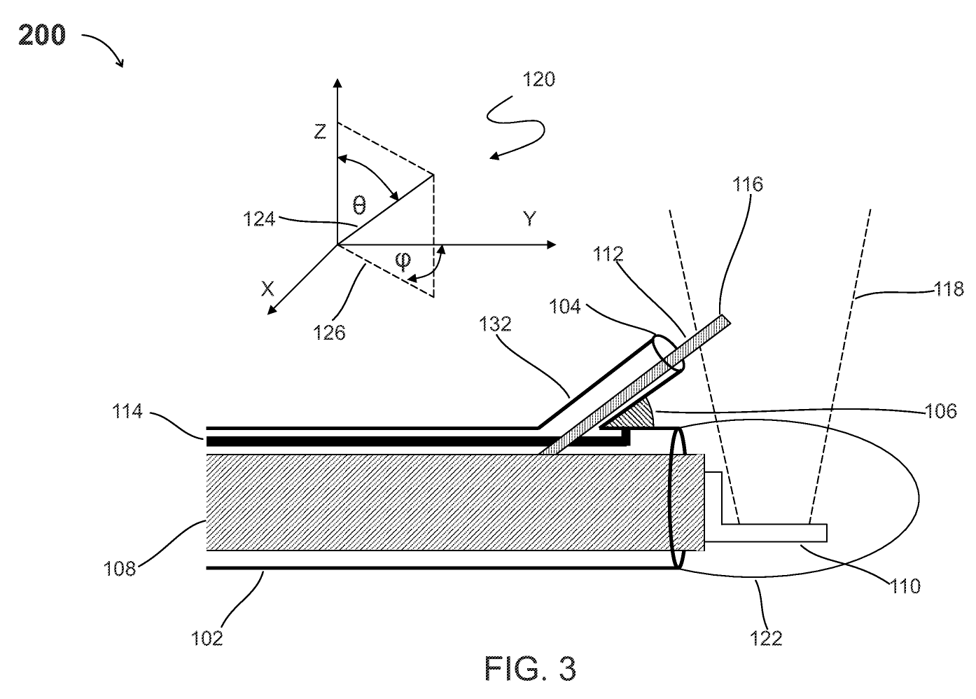

FIG. 3 is a schematic drawing illustrating an endoscope sheath featuring a sheath channel for housing an endoscopic tool, according to some embodiments of the invention.

Apparatus 200 has the same elements as apparatus 100 of FIG. 1A and/or of apparatus 101 of FIG. 1B, and further includes a sheath channel 132, for housing endoscopic tool 112. Sheath channel 132 has an opening 104, configured for being traversed by endoscopic tool 122. Therefore, distal tip 116 of endoscopic tool 112 is not housed within sheath channel 132, as distal tip 116 is configured for coming into direct contact with a target inside a body lumen, for probing, treating, and/or manipulating the target.

Optionally, sheath channel 132 is an integral part of endoscope housing 102, protruding from endoscope housing 102. Alternatively, sheath channel 132 may be an integral part of a tool channel 117. (Tool channel 117 is shown in FIG. 1B.) Optionally, sheath channel 132 is a detachable element that may be fitted onto endoscope housing 102 (or tool channel 117), according to a user's need.

Optionally, tool deflector 106 does not touch any part of endoscopic tool 112. Rather, tool deflector 106 changes the orientation of endoscopic tool 112, by moving sheath channel 132, which is in direct contact with endoscopic tool 112, for example, by encircling endoscopic tool 112. This configuration may reduce the risk of tool deflector 106 being damaged by contact with a sharp edge of endoscopic tool 112. Optionally, tool deflector 106 is in direct contact with endoscopic tool 112, and changes the orientation of endoscopic tool 112 by direct contact, without acting on sheath channel 132.

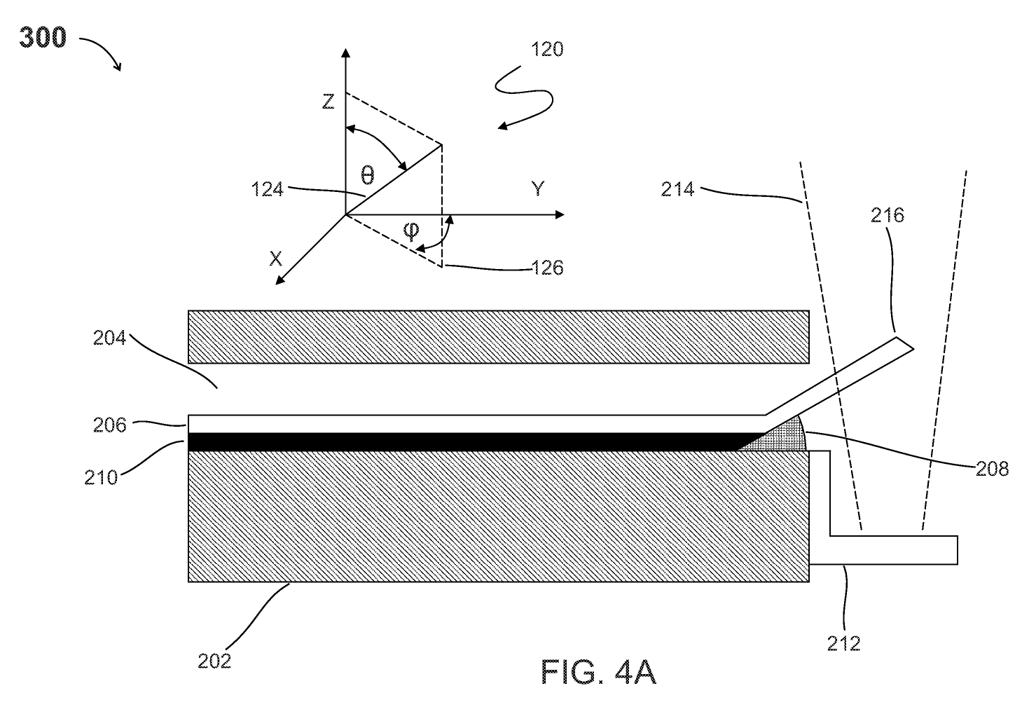

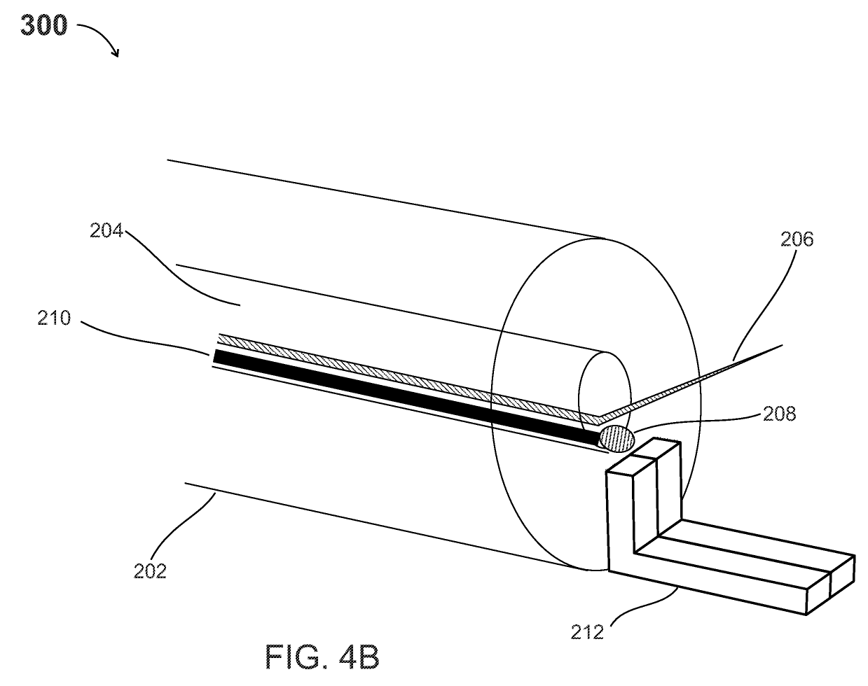

FIGS. 4a and 4b are schematic drawings illustrating an insertion tube of an endoscope featuring a tool deflector located at a distal end of an endoscopic channel, according to an embodiment of the invention. FIG. 4a is a cross section view of an insertion tube 202. FIG. 4b is an isometric view of insertion tube 202.

Optionally, the endoscope is a bronchoscope. Optionally the bronchoscope is characterized by a rigid insertion tube, configured, for example, for removing objects that have become obstructed in the airways of a patient. Optionally the bronchoscope is characterized by a flexible insertion tube, configured, for example, for reaching remote areas within the airways and probing and/or treating the above areas.

In apparatus 300, insertion tube 202 is characterized by an endoscopic channel 204, and an inflatable tool deflector 208. Endoscopic channel 204 is an open lumen which traverses insertion tube 202 along its length, from a proximal extremity thereof to a distal extremity thereof and is configured for housing an endoscopic tool 206. A distal tip 216 of endoscopic tool 206 is not housed within endoscopic channel 204, as tip 216 is configured to come into direct contact with a target in the body lumen, for probing, treating, and/or manipulating the target. Inflatable tool deflector 208 is placed at the distal end of endoscopic channel 204 and is in contact with endoscopic tool 206. Inflatable tool deflector 208 is configured to be controlled by a user in order to change the orientation of endoscopic tool 206. Optionally, apparatus 300 further includes a conduit 210, the distal end of which is attached to tool deflector 208, for moving fluid into or out of tool deflector 208. Optionally, conduit 210 is housed within endoscopic channel 204. Optionally, tool deflector 208 comprises a two or more independently inflatable sub-units, as described above with respect to deflector 106, and optionally conduit 210 comprises two or more sub-conduits for moving fluid to and from those independently inflatable sub-units, as described above with reference to conduit 114.

Referring now to a coordinate system 120, Y is defined to be the direction of movement of a distal end of insertion tube 202, and X and Z are constructed to be normal to Y and to each other. Direction 124 is an orientation of endoscopic tool 206. The orientation of endoscopic tool 206 is defined by an angle θ formed between direction 124 and the Z axis, and an angle φ formed between a projection 126 of direction 124 upon the XY plane and the Y axis. Tool deflector 208 changes the direction of movement of endoscopic tool 206, by changing either angle θ, angle φ, or a both angles, as described above with respect to tool deflector 106. Direction 124 may also be influenced by rotating insertion tube 202.

Inflatable tool deflector 208 includes at least one inflatable unit, and moves endoscopic tool 206 by direct contact. Optionally, inflatable tool deflector 208 may be inflated and deflated with the fluids, through the control unit, and according to the methods illustrated above. Inflatable tool deflector 208 is optionally made of materials listed above.

Optionally, insertion tube 202 is configured to hold an imaging sensor 212 at the distal end of insertion tube 202. Optionally, insertion tube 202 is configured to hold more than one imaging sensor. Optionally, imaging sensor 212 is an ultrasound imaging sensor, a CCD sensor, a CMOS sensor, a MEI sensor, or a fluorescent imager sensor. Imaging sensor 212 has a field of view 214. Optionally, endoscope 202 is configured so that at least distal tip 216 of endoscopic tool 206 is within field of view 214. Optionally, insertion tube 202 includes optical fibers, as commonly found in bronchoscopes, for conveying an image of the body lumen to a user. Optionally, insertion tube 202 holds a light source—for example a light emitting diode (LED)—in order to illuminate the body lumen and improve the quality of the image seen by the user.

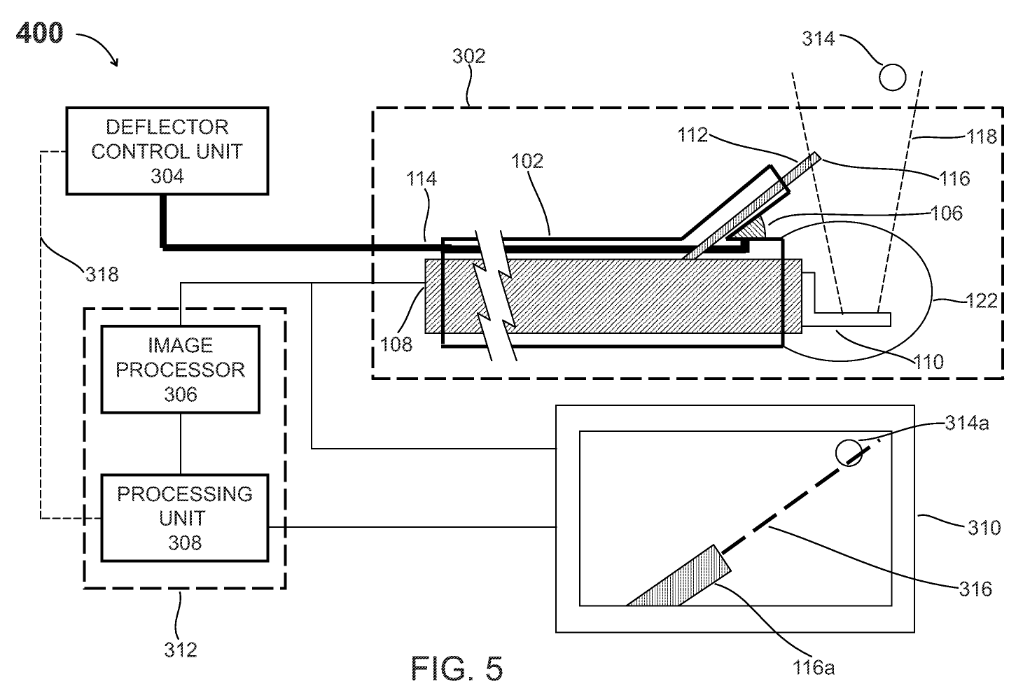

FIG. 5 is a schematic drawing illustrating a system 400 for guiding an endoscopic tool 112 associated with an endoscope toward a target 314 within a body lumen, with the aid of an image processor 306, according to a preferred embodiment of the invention.

System 400 includes an endoscopic apparatus 302, a deflector control unit 304, image processor 306, a processing unit 308, and a screen 310. Optionally, image processor 306 and processing unit 308, are comprised within a computer 312. Optionally, processing unit 308 is the processing unit of computer 312.

Endoscopic apparatus 302 includes an endoscope having an insertion tube 108, an endoscopic tool 112, an imaging sensor 110 having a field of view 118, and a tool deflector 106. Optionally, endoscopic apparatus 302 includes more than one imaging sensor. Optionally, tool deflector 106 is part of an endoscope sheath, as depicted in FIGS. 1, 2, and 3, and endoscopic apparatus 302 may be substituted by one of apparatus 100 shown in FIG. 1, apparatus 150 shown in FIG. 2, or by apparatus 200, shown in FIG. 3. Optionally, tool deflector 106 is part of the endoscope having insertion tube 202, as shown in FIGS. 4a and 4b, in which case, endoscopic apparatus 302 is substituted by apparatus 300, depicted in FIGS. 4a and 4b.

Optionally, tool deflector 106 includes an inflatable unit, as described above. Optionally, tool deflector 106 is inflated and deflated by the movement of a fluid into and out of tool deflector 106 through a conduit 114. Optionally, deflector control unit 304 connected to conduit 114 is a cylinder-piston device or a variable pressure regulator, as described above, in the description of FIG. 1.

In apparatus 400, imaging sensor 110, attached to a distal end of insertion tube 108, generates an image of the body lumen, which includes a distal tip 116 of endoscopic tool 112, and a target 314 for endoscopic tool 112 to reach. The image of target 314 is 314a, and the image of distal tip 116 is 116a. Optionally, imaging sensor 110 is an ultrasound imaging sensor, configured for providing an ultrasound image of the body lumen. Optionally imager 110 is a CCD sensor, a CMOS sensor, a MEI sensor, or a fluorescent imager sensor.

A signal is sent by endoscopic apparatus 302 to screen 310, where the image is displayed. The same signal is sent by endoscopic apparatus 302 to image processor 306. Image processor 306 analyzes the image and calculates the orientation of endoscopic tool 112, optionally through image analysis algorithms. The calculated endoscopic tool orientation value is sent to processing unit 308, which uses the orientation value to calculate the future trajectory of endoscopic tool 112. Optionally, if deflector control unit 304 includes an electronically controlled linear actuator or a variable pressure regulator, a further signal 318 is sent from deflector control unit 304 to processing unit 308, for obtaining more precise trajectory calculations. Optionally, signal 318 is sent by deflector control unit 304 and contains data, such as pressure values, of the fluid directed to tool deflector 106. Optionally, the data of signal 318 is related to an orientation and/or a change in orientation of endoscopic tool 112, for example through a calibration process of tool deflector 106. The trajectory data is sent by processing unit 308 to screen 310, and is displayed as a trajectory track 316, superimposed in real time upon the image generated by imaging sensor 110.

Deflector control unit 304 allows a user to control tool deflector 106, and therefore to change the orientation of endoscopic tool 112. As the user changes the orientation of endoscopic tool 112, the new orientation of endoscopic tool 112 is instantaneously calculated by image processor 306, and therefore a new trajectory track 316 is instantaneously calculated and displayed on screen 310. Once trajectory track 316 crosses image 314a of target 314 on screen 310, the user may push insertion tube 108 and/or endoscopic tool 112, farther into the lumen, towards target 314. Since trajectory track 316 is displayed on screen 310 in real time, changes in the orientation of endoscopic tool 112 can be readily corrected for, by using tool deflector 106 to change the orientation of endoscopic tool 112 so that trajectory track 316 crosses image 314a of target 314. In a preferred embodiment of the invention, tool deflector 106 is inflatable and is inflated and deflated by the movement of a fluid through conduit 114.

Optionally, tool deflector 106 is inflated and deflated by methods that do not require the presence of conduit 114. An exemplary method described above involves expanding—for example, through heating—a fluid that is inside tool deflector 106. Optionally, tool deflector 106 changes the orientation of endoscopic tool 112 by methods other than inflation and deflation. For example, tool deflector 106 may be a cam type deflector, or an electromechanical deflector controlled by electrical signals.

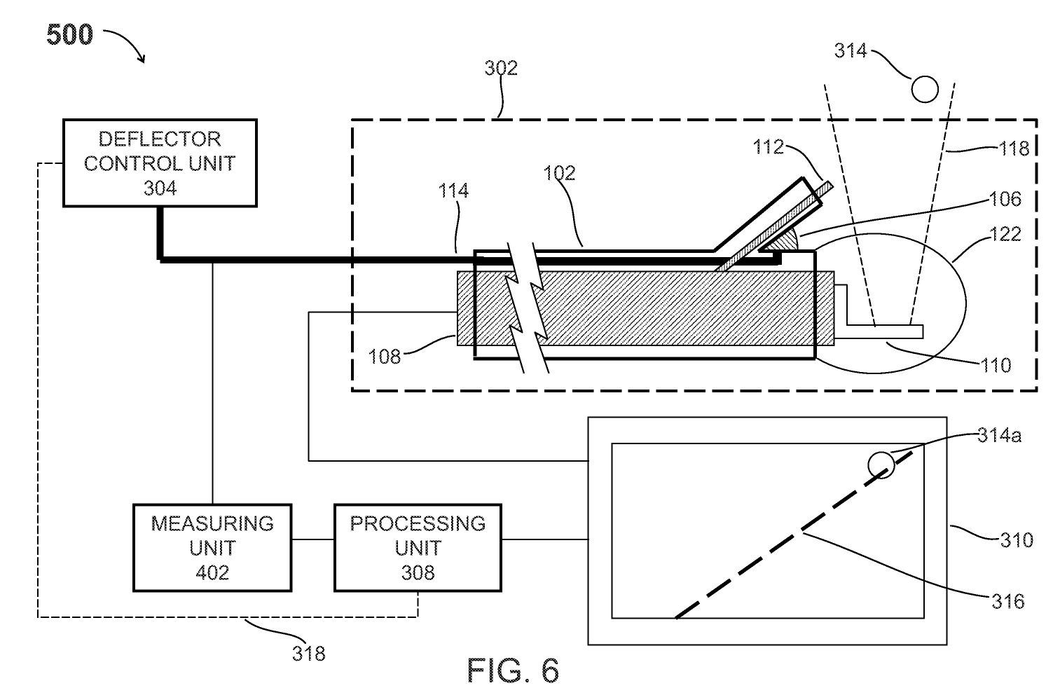

FIG. 6 is a schematic drawing illustrating a system for guiding an endoscopic tool 112 associated with an endoscope toward a target 314 within a body lumen, without the assistance of image processing, according to an alternative embodiment of the invention.

Apparatus 500 includes an endoscopic apparatus 302, a deflector control unit 304, a measuring unit 402, a processing unit 308, and a screen 310. Endoscopic apparatus 302 is the same as endoscopic apparatus 302 described in FIG. 5, except for the fact that endoscopic tool 112 is not necessarily within field of view 118 of imaging sensor 110.

In apparatus 500, measuring unit 402 is connected to conduit 114, and measures the fluid flow into or out of tool deflector 106. Optionally, measuring unit 402 measures the pressure of the fluid within conduit 114. Optionally measuring unit 402 is connected to tool deflector 106 and measures the volume and/or the pressure of fluid within tool deflector 106, or within independently inflatable sub-units of deflector 106. Optionally, measuring unit 402 measures one, more, or all of the fluid flow into and out of tool deflector 106, the fluid pressure within measuring unit 114, the fluid pressure within tool deflector 106, and the fluid volume within tool deflector 106 and/or sub-units thereof. Optionally, measuring unit 402 is included within deflector control unit 304.

Processing unit 308 receives measured values from measuring unit 402, and calculates the orientation of endoscopic tool 112, according to data from a calibration of tool deflector 106, which relates the measured values to an orientation or an orientation change of endoscopic tool 112. Processing unit 308 also calculates the trajectory of endoscopic tool 112, according to the orientation of endoscopic tool 112, and sends a signal to screen 310. Optionally, if deflector control unit 304 includes an electronically controlled linear actuator or a variable pressure regulator, a further signal 318 is sent from deflector control unit 304 to processing unit 308, for obtaining more precise trajectory calculations, as explained above. At screen 310, the signal from processing unit 308 is converted into trajectory track 316, which is used for guiding the endoscopic tool towards target 314.

Optionally, apparatus 500 further includes an image processor, as shown in FIG. 5 and described above, and trajectory track 316 is calculated both through image processing and the measurement of properties of the fluids directed to tool deflector 106.

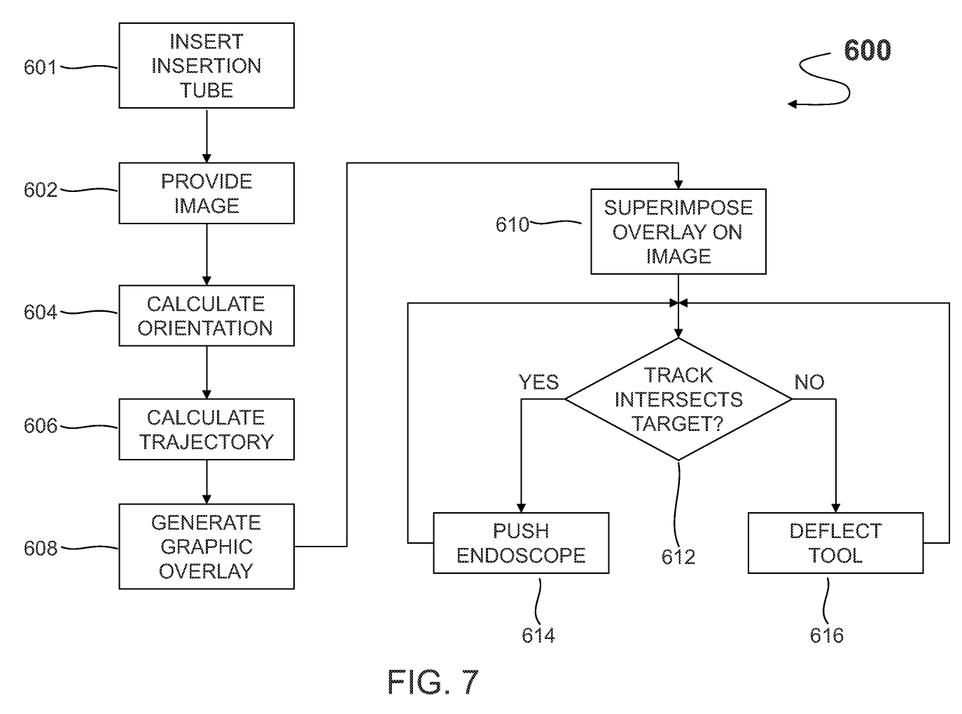

FIG. 7 is a flowchart illustrating a method 600 for guiding an endoscopic tool associated with an endoscope toward a target within a body lumen, according to some embodiments of the invention.

At 601, an insertion tube of an endoscope is inserted into the body lumen.

At 602, an image of the body lumen is provided by one or more imaging sensors which are associated with the endoscope. Optionally, one or more imaging sensors are located at a distal extremity of the insertion tube. The image of the body lumen includes at least the target within the body lumen. Optionally, the image also includes a distal tip of the endoscopic tool. Optionally, the image is provided by a sensor of an ultrasound imager. Optionally, the imaging sensor is controlled by a user through a computer. Optionally, a user identifies a treatment target within the image of the body lumen, optionally by marking or delimiting a portion of that image using a graphical user interface. Optionally, an image processing algorithm identifies a treatment target algorithmically and provides a graphical indication of that target's position on the body lumen image, and optionally a user inspects that graphically marked image and confirms or rejects that algorithmically generated target determination.

At 604, the orientation of the endoscopic tool is calculated. Optionally, image processing algorithms are used to identify and track the endoscopic tool. The orientation of the endoscopic tool is therefore calculated through the analysis of the image of the distal tip of the endoscopic tool, for example by an image processor included within a computer controlling the imaging sensor. In such case, the calculation is a standard trigonometric calculation.

Optionally, the orientation is calculated through measurements of one or more properties of fluids directed to the tool deflector, coupled with data from a calibration of the tool deflector, as illustrated above. Optionally, the above properties are the flow and/or pressure of the fluid moving into and out of the tool deflector. Optionally, the above properties are the volume and/or pressure of the fluid inside the tool deflector.

At 606, the trajectory of the endoscopic tool is calculated, based on the endoscopic tool's orientation.

At 608, a graphic overlay of the trajectory is generated. The graphic overlay of the trajectory is herein also referred to as “trajectory track”.

At 610, the graphic overlay of the trajectory is superimposed on the image generated at 602.

At 612, the graphic overlay of the trajectory is observed: if the overlay intersects the image of the target, the endoscopic tool is advanced, by being pushed farther into the lumen, towards the target, if desired, at 614; optionally or alternatively, the insertion tube of the endoscope is pushed farther into the lumen in order to direct endoscopic tool towards the target; the graphic overlay of the trajectory of the endoscopic tool is constantly monitored so that the overlay continues to intersect the image of the target, while the endoscope and endoscopic tool are moved; if the overlay does not intersect the image of the target, the endoscopic tool is deflected at 616, until it does, and then directed towards the target at 614.

In some embodiments of the invention, the observation at 612 is aided by visual and/or audible feedback. Optionally, a processor may be used to determine whether the overlay intersects the image of the target, which target image may have been identified by a user or algorithmically determined by image interpretation software and optionally confirmed by a user, as explained above. Optionally, the processor changes the color of the overlay. For example, the overlay may be red when the overlay does not intersect the image of the target, and green when the overlay intersects the image of the target. Optionally, the color changes gradually, as the overlay approaches the image of the target.

Optionally or alternatively, the processor is connected to one or more speakers, and instructs the speakers to emit different sounds, depending on whether the overlay intersects the image of the target, to inform a user about the orientation of the tool deflector with respect to the image of the target. For example, the speakers may emit a repeating sound characterized by a time interval between repetitions, and the time interval becomes shorter as the overlay moves closer to the image of the target. When the overlay crosses the image of the target, the time interval becomes null, and a constant sound is emitted. Optionally, the processor is a computer.

It is expected that during the life of a patent maturing from this application many relevant algorithms for calculating the orientation of the tool deflector will be developed and the scope of the term a processor and user interface are intended to include all such new technologies a priori.

The terms “comprises”, “comprising”, “includes”, “including”, “having” and their conjugates mean “including but not limited to”.

As used herein, the singular form “a”, “an” and “the” include plural references unless the context clearly dictates otherwise. For example, the term “a compound” or “at least one compound” may include a plurality of compounds, including mixtures thereof.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. An endoscope sheath, comprising:
an endoscope housing, configured for housing at least a distal end of an insertion tube of an endoscope and allowing the traversing of an endoscopic tool therethrough; and
a tool deflector configured for deflecting said endoscopic tool by changing a direction of movement of said endoscopic tool in relation to a direction of movement of said distal end of said insertion tube.

2. The endoscope sheath of claim 1, wherein said insertion tube belongs to a bronchoscope.

3. The endoscope sheath of claim 1, wherein said endoscope housing is configured for housing the whole endoscope.

4. The endoscope sheath of claim 1, configured for being used only once.

5. The endoscope sheath of claim 1, wherein said endoscopic tool traverses said endoscope housing through at least one opening located on a surface of said endoscope housing.

6. The endoscope of claim 1, wherein said at least one opening is located at a distal extremity of at least one sheath channel, said sheath channel being configured for housing at least a portion of said endoscopic tool.

7. The endoscope sheath of claim 1, wherein said tool deflector deflects said endoscopic tool in a plurality of directions.

8. The endoscope sheath of claim 1, wherein said tool deflector is located on a surface of said endoscope housing, between a point at which said endoscopic tool traverses said endoscope housing and a distal extremity of said endoscope housing.

9. The endoscope sheath of claim 1, wherein said tool deflector is located on one of: an inner surface of said endoscope housing, and an outer surface of said endoscope housing.

10. The endoscope sheath of claim 6, wherein said at least one sheath channel is an integral part of said endoscope housing, and protrudes from said endoscope housing.

11. The endoscope sheath of claim 6, wherein said sheath channel is detachable from said endoscope housing.

12. The endoscope sheath of claim 6, wherein said tool deflector is configured to change said orientation of said endoscopic tool by moving a distal end of said sheath channel.

13. The endoscope sheath of claim 1, wherein said tool deflector is inflatable and deflects said distal end of said endoscopic tool by expanding and contracting.

14. The endoscope sheath of claim 13, further including a conduit for moving at least one fluid into and out of said tool deflector.

15. The endoscope sheath of claim 13, wherein a fluid within said tool deflector is heated to expand, and cooled to contract, thereby expanding and contracting said tool deflector.

16. The endoscope sheath of claim 8, wherein said point at which said sheath housing is traversed by said endoscopic tool is positioned such that at least one tip of said endoscopic tool is within a field of view of at least one imaging sensor attached to a distal end of said insertion tube.

17. The endoscope sheath of claim 16, wherein said at least one imaging sensor is selected from a group of an ultrasound imaging sensor, a charged couple device (CCD) sensor, and a complementary metal oxide semiconductor (CMOS) sensor, a magnetic endoscopic imager (MEI) sensor, and a fluorescent imager.

18. An endoscope, comprising:
an endoscopic channel, traversing an insertion tube of the endoscope, and configured for housing at least one endoscopic tool; and
an inflatable tool deflector located at a distal end of said endoscopic channel, for gradually deflecting a distal end of said endoscopic tool in relation to a distal end of said insertion tube;
wherein said inflatable tool deflector is in contact with said at least one endoscopic tool, and a change in at least one property of said inflatable tool deflector causes a deflection of said distal end of said endoscopic tool, with respect to said distal end of said insertion tube, by changing a direction of movement of said distal end of said endoscopic tool in relation to a direction of movement of said distal end of said insertion tube.

19. The endoscope of claim 18, wherein the endoscope is configured for being inserted into the airways of a patient.

20. The endoscope of claim 18, wherein said property of said inflatable tool deflector is one of volume and shape.

21. The endoscope of claim 18, further comprising at least one imaging sensor, for providing an image of at least a body lumen within which said insertion tube is inserted.

22. The endoscope of claim 21, wherein said at least one imaging sensor is selected from a group of an ultrasound imaging sensor, a charged couple device (CCD) sensor, and a complementary metal oxide semiconductor (CMOS) sensor, a magnetic endoscopic imager (MEI) sensor, and a fluorescent imaging sensor.

23. The endoscope of claim 21, wherein a distal tip of said at least one endoscopic tool housed in said endoscopic channel is within a field of view of said at least one imaging sensor, so that an image of said distal tip is provided by said at least one imaging sensor.

24. The endoscope of claim 23, configured to be connected to an image processor, which determines said orientation of said endoscopic tool, through an analysis of said image of said tip.

25. A system for generating a trajectory track of an endoscopic tool associated with an endoscope within a body lumen, comprising:
at least one imaging sensor attached to a distal end of an insertion tube of the endoscope, for generating an image of the body lumen;
a processing unit, for calculating the trajectory of the endoscopic tool based on an orientation of the endoscopic tool; and
a screen, for displaying the trajectory track on said image.

26. The system of claim 25, wherein said image of the body lumen further includes a tip on a distal side of the endoscopic tool.

27. The system of claim 26, further including:
an image processing unit, for calculating said orientation of the endoscopic tool, based on said image from said at least one imaging sensor.

28. The system of claim 25, wherein said at least one imaging sensor is selected from a group of an ultrasound imaging sensor, a charged couple device (CCD) sensor, and a complementary metal oxide semiconductor (CMOS) sensor, a magnetic endoscopic imager (MEI) sensor, and a fluorescent imaging sensor.

29. The system of claim 25, further comprising:
a tool deflector, for changing said orientation of the endoscopic tool, relative to the endoscope; and
a measuring unit, for measuring at least one property of said tool deflector;
wherein said processing unit receives said measured property from said measuring unit, and calculates said orientation of the endoscopic tool, according to said property.

30. The system of claim 29, wherein said tool deflector comprises at least one inflatable unit, and said measuring unit measures one or more of:
a flow of fluid into and out of said inflatable unit of said tool deflector;
a pressure of said fluid; and
a volume of said fluid within said tool deflector.

31. A system for guiding an endoscopic tool associated to an endoscope toward a target within a body lumen, comprising:
the system of claim 25, wherein said at least one imaging sensor generates said image that further includes the target; and
a tool deflector, for changing said orientation of the endoscopic tool toward the target.

32. A method for changing an orientation of an endoscopic tool associated with the endoscope, relative to a distal end of an insertion tube of the endoscope, comprising:
inserting the insertion tube into a body lumen; and
changing the orientation of the endoscopic tool by changing at least one property of an inflatable tool deflector in contact with the endoscopic tool.

33. The method of claim 32, wherein said property is one of volume and shape.

34. The method of claim 32, further comprising:
providing an image of a target within said body lumen; and
calculating the orientation of the endoscopic tool.

35. The method of claim 34, wherein said calculating is performed through at least one of:
an analysis of an image of the endoscopic tool, by an image processor; and
a measurement of at least one property of at least one fluid directed to said inflatable tool deflector, and a conversion of said property into said orientation, according to calibration data.

36. The method of claim 34, further comprising:
calculating an estimated trajectory of the endoscopic tool, based on the orientation; and
superimposing a graphical trajectory track of said trajectory on said image.

37. The method of claim 36, further comprising:
changing the orientation of the endoscopic tool so that said graphical trajectory track crosses said image of said target;
advancing the insertion tube and/or the endoscopic tool farther into said body lumen;
observing whether said graphical trajectory track crosses said image of said target; and
changing the orientation of the endoscopic tool, if needed.

38. The method of claim 37, wherein said observing is performed by a processing unit, and further including one or more of:
assigning a color to said graphical trajectory track, according to the orientation of said graphical trajectory track relative to said image of said target; and
emitting a sound, according to the orientation of said graphical trajectory track relative to said image of said target.
